(19) United States
(12) Patent Application Publication
(10) Pub. No.: US 2024/0407935 A1
(43) Pub. Date: Dec. 12, 2024

(54) Stent Delivery Systems with Enhanced Accuracy

(71) Applicant: Boston Scientific Scimed, Inc., Maple Grove, MN (US)

(72) Inventors: Michael William Nagel, Andover, MN (US); Brady Scott Logan, Maple Grove, MN (US); Thomas Skoog, Elk River, MN (US)

(73) Assignee: Boston Scientific Scimed, Inc., Maple Grove, MN (US)

(21) Appl. No.: 18/735,346

(22) Filed: Jun. 6, 2024

Related U.S. Application Data
(60) Provisional application No. 63/471,612, filed on Jun. 7, 2023.

(51) Int. Cl. A61F 2/966; A61F 2/95

(52) U.S. Cl. CPC A61F 2/966; A61F 2/9517

(57) ABSTRACT

Stent delivery systems as well as methods for making and using stent delivery systems are disclosed. An example stent delivery system may include an elongate shaft including an inner member having a stent receiving region, a deployment sheath slidably disposed along the inner member, and an intermediate shaft disposed between the inner member and the deployment sheath. The intermediate shaft may have an outer diameter. A handle may be coupled to the elongate shaft. A rack member may be coupled to the deployment sheath. At least a region of the rack member may be configured to extend within the handle. The rack member may have an axial slot formed therein. At least a portion of the axial slot may have a width that is less than the outer diameter of the intermediate shaft.

### CROSS-REFERENCE TO RELATED APPLICATIONS

This application claims the benefit of priority under 35 U.S.C. § 119 of U.S. Provisional Application No. 63/471,612, filed Jun. 7, 2023, the entire disclosure of which is hereby incorporated by reference.

### TECHNICAL FIELD

The present disclosure pertains to medical devices, and methods for manufacturing medical devices. More particularly, the present disclosure pertains to stent delivery systems

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A stent delivery system is disclosed. The stent delivery system comprises: an elongate shaft including an inner member having a stent receiving region, a deployment sheath slidably disposed along the inner member, and an intermediate shaft disposed between the inner member and the deployment sheath; wherein the intermediate shaft has an outer diameter; a handle coupled to the elongate shaft; a rack member coupled to the deployment sheath, wherein at least a region of the rack member is configured to extend within the handle; wherein the rack member has an axial slot formed therein; and wherein at least a portion of the axial slot has a width that is less than the outer diameter of the intermediate shaft.

Alternatively or additionally to any of the embodiments above, the rack member includes a plurality of teeth.

Alternatively or additionally to any of the embodiments above, further comprising a pull handle coupled to the rack member.

Alternatively or additionally to any of the embodiments above, the pull handle is disposed proximal of a proximal end of the handle.

Alternatively or additionally to any of the embodiments above, the portion of the axial slot having the width that is less than to the outer diameter of the intermediate shaft is disposed adjacent to the pull handle.

Alternatively or additionally to any of the embodiments above, a proximal portion of the axial slot having a second width that is greater than or equal to the outer diameter of the intermediate shaft is disposed between the pull handle and the portion of the axial slot having the width that is less than the outer diameter of the intermediate shaft.

Alternatively or additionally to any of the embodiments above, the rack member has a central bore having a bore diameter and wherein the width of the portion of the axial slot is less than to the central bore.

Alternatively or additionally to any of the embodiments above, the rack member has one or more molding apertures formed therein.

Alternatively or additionally to any of the embodiments above, the rack member has a sidewall opening formed therein that is configured to allow the deployment sheath to be mechanically secured to the rack member.

Alternatively or additionally to any of the embodiments above, the elongate shaft includes an outer shaft disposed along a section of an outer surface of the deployment sheath.

A stent delivery system is disclosed. The stent delivery system comprises: an inner member having a stent receiving region; a deployment sheath slidably disposed along the inner member; a rack member coupled to the deployment sheath, the rack member having an axial slot formed therein; wherein the axial slot has a first portion having a first slot width and a second portion having a second slot width different from the first slot width; and a handle disposed over at least a portion of the rack member.

Alternatively or additionally to any of the embodiments above, further comprising an intermediate shaft disposed between the inner member and the deployment sheath.

Alternatively or additionally to any of the embodiments above, the first slot width is greater than or equal to an outer diameter of the intermediate shaft.

Alternatively or additionally to any of the embodiments above, the second slot width is less than an outer diameter of the intermediate shaft.

Alternatively or additionally to any of the embodiments above, further comprising a pull handle coupled to the rack member and disposed proximal of a proximal end of the handle.

Alternatively or additionally to any of the embodiments above, the first portion of the axial slot is disposed adjacent to the pull handle.

Alternatively or additionally to any of the embodiments above, the first portion of the axial slot is disposed between the pull handle and the first portion of the axial slot.

Alternatively or additionally to any of the embodiments above, the rack has a central bore having a bore diameter and wherein the second slot width of the axial slot is less than the central bore.

A stent delivery system is disclosed. The stent delivery system comprises: an inner member having a stent receiving region; a deployment sheath slidably disposed along the inner member; an intermediate shaft disposed between the inner member and the deployment sheath; a rack member coupled to the deployment sheath, the rack member having an axial slot formed therein; and wherein the axial slot has a first portion having a first slot width and a second portion having a second slot width different from the first slot width.

Alternatively or additionally to any of the embodiments above, the second slot width is less than an outer diameter of the intermediate shaft.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is side view of an example system.

FIG. 2 is a side view of a portion of an example system.

FIG. 3 is a partial cross-sectional side view of a portion of an example system.

FIG. 4 is a partial cross-sectional side view of a portion of an example system.

FIG. 5 is a partial cross-sectional side view of a portion of an example system.

FIG. 6 is a side view of a portion of an example system.

FIG. 7 is a perspective view of a portion of an example system.

FIG. 8 is a side view of a portion of an example system.

FIG. 9 is a side view of a portion of an example system.

FIG. 10 is a partial cross-sectional side view taken through line 10-10 in FIG. 9.

FIG. 11 is a partial cross-sectional side view taken through line 11-11 in FIG. 9.

FIG. 12 is a side view of a portion of an example system.

FIG. 13 is a side view of a portion of an example system.

FIG. 14 is a side view of a portion of an example system.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

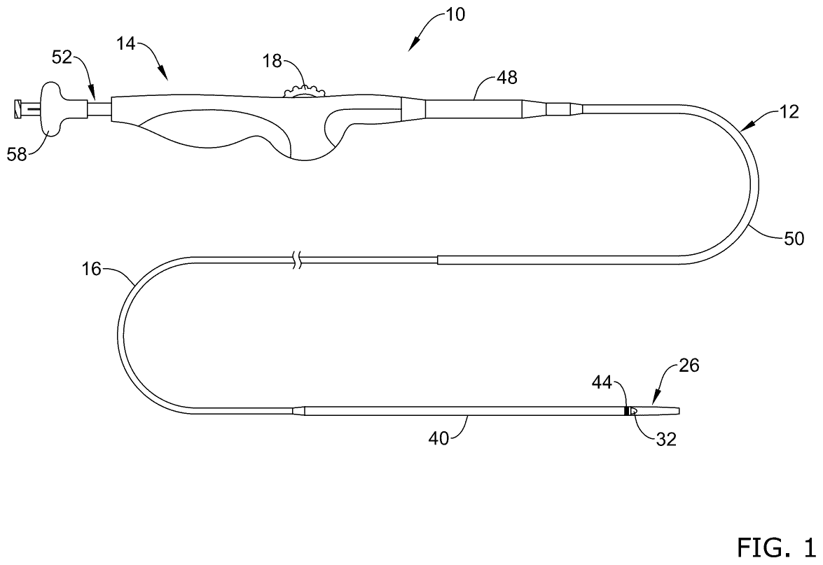

FIG. 1 illustrates an example stent delivery system 10. The system 10 may include an elongate shaft 12 and a handle 14 coupled to the shaft 12. In general, the system 10 may be used to deliver a suitable stent, graft, endoprosthesis, and/or the like to an area of interest within a body lumen of a patient. The body lumen may be a blood vessel located near the heart (e.g., within or near a cardiac vessel), within a peripheral vessel, within a neurological vessel, or at any other suitable location. Deployment of the stent may include the proximal retraction of a deployment sheath 16, which overlies the stent. Retraction of the deployment sheath 16 may include the actuation of an actuation member 18 generally disposed at the handle 14. In the example illustrated in FIG. 1, the actuation member 18 is a thumbwheel that can be rotated by a clinician in order to accomplish proximal retraction of the deployment sheath 16. Numerous other actuation members are contemplated. A number of other structures and features of the system 10 can be seen in FIG. 1 and are labeled with reference numbers. Additional discussion of some of these structures can be found below.

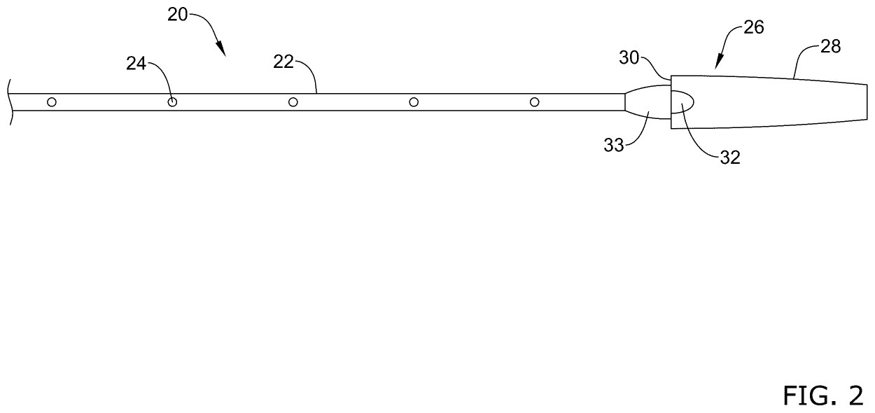

FIGS. 2-6 illustrate examples of some of the structural components that may be included as a part of the system 10. For example, the system 10 may include an inner shaft or member 20 as illustrated in FIG. 2. In at least some embodiments, the inner member 20 may be a tubular structure and, thus, may include a lumen (not shown). The lumen may be a guidewire lumen that extends along at least a portion of the length of the inner member 20. Accordingly, the system 10 may be advanced over a guidewire to the desired target location in the vasculature. In addition, or in alternative embodiments, the lumen may be a perfusion/aspiration lumen that allows portions, components, or all of the system 10 to be flushed, perfused, aspirated, or the like.

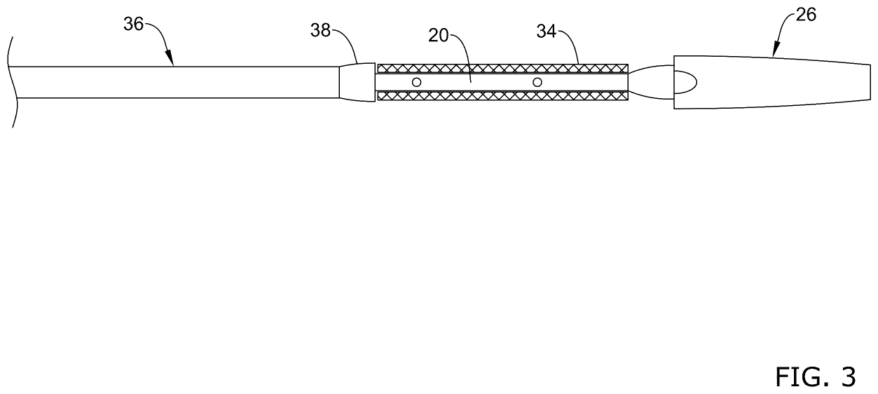

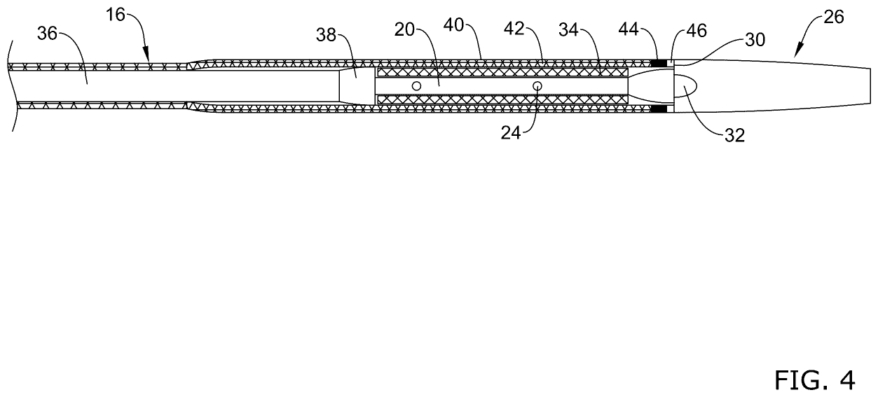

The inner member 20 may include a stent receiving region 22 about which a stent (not shown, can be seen in FIGS. 3-4) may be disposed. The length and/or configuration of the stent receiving region 22 may vary. For example, the stent receiving region 22 may have a length sufficient for the stent to be disposed thereon. It can be appreciated that as the length of the stent utilized for the system 10 increases, the length of the stent receiving region 22 also increases.

Along or otherwise disposed adjacent the stent receiving region 22 may be one or more perfusion ports 24. The ports 24 may extend through the wall of the inner member 20 such that fluid may be infused through the lumen of the inner member 20 and may be flushed through the ports 24. This may be desirable for a number of reasons. For example, the ports 24 may allow a clinician to evacuate air bubbles that may be trapped adjacent the stent by perfusing fluid through the ports 24. In addition, the ports 24 may be used to aspirate fluid that may be disposed along the inner member 20. The ports 24 may also aid in sterilization and/or other preparatory processing steps that may be involved in preparing the system 10 for use.

A tip 26 may be attached to or otherwise disposed at the distal end of the inner member 20. The tip 26 may generally have a rounded or smooth shape that provides a generally atraumatic distal end to the system 10. For example, the tip 26 may have a smooth tapered distal portion 28 that gently tapers. The tip 26 may also include a proximal ridge 30 that is configured so that the deployment sheath 16 can abut therewith. The tip 26 may also include a tapered proximal portion 33. Numerous other shapes and/or configurations are contemplated for the tip 26.

The tip 26 may also include one or more cutouts or flats 32 formed therein. For the purposes of this disclosure, the flats 32 are understood to be cutouts or flattened portions of the tip 26 where the outer dimension or profile of the tip 26 is reduced. The name “flats” comes from the fact that these regions may have a somewhat “flat” appearance when compared to the remainder of the tip 26, which generally may have a rounded profile. The shape, however, of the flats 32 is not meant to be limited to being flat or planar as numerous shapes are contemplated.

The flats 32 may allow for a gap or space to be defined between the inner member 20 and the deployment sheath 16 when the deployment sheath 16 abuts the proximal ridge 30 of the tip 26. This gap may allow for fluid, for example perfusion fluid passed through the ports 24, to flow out from the deployment sheath 16. Thus, the flats 32 may be used in conjunction with the ports 24 to allow portions or all of the system 10 to be flushed or otherwise evacuated of air bubbles.

FIG. 3 illustrates the inner member 20 with some additional structure of the system 10. In this figure, a stent 34 is disposed about the inner member 20 (e.g., about the stent receiving region 22 of the inner member 20). In some embodiments, the stent 34 is a self-expanding stent. Accordingly, the stent 34 may be biased to outwardly expand. Because of this, stent 34 may not be “loaded onto” the inner member 20 in a strict sense but rather may be thought of as being disposed about or surrounding the inner member 20. The stent 34 may then be restrained within the deployment sheath 16. In alternative embodiments, however, the stent 34 may be directly loaded onto the inner member 20 via crimping or any other suitable mechanical holding mechanism.

An intermediate tube 36 may also be disposed over the inner member 20. In at least some instances, the intermediate tube 36 may extend from a position adjacent to the proximal end of the inner member 20 to a position proximal of the distal end of the inner member 20. The intermediate tube 36 may include a bumper 38. In practice, the bumper 38 may function by preventing any unwanted proximal movement of the stent 34 during navigation and/or deployment of the stent 34.

The bumper 38 may have any suitable form. In some instances, the bumper 38 may be defined by a relatively short tube or sleeve that is disposed about the intermediate tube 36. The material utilized for the sleeve may be the same or different from that of the intermediate tube 36. The intermediate tube 36 may have a tapered or otherwise smooth transition in outer diameter adjacent the bumper 38. For example, polymeric material may be disposed or reflowed adjacent the bumper 38 (which may include disposing the polymeric material about a portion or all of the bumper 38) so as to define a gentle transition in outer diameter at the bumper 38. Other configurations are contemplated and may be utilized in alternative embodiments.

FIG. 4 illustrates additional structure of the system 10. Here the deployment sheath 16 can be seen disposed over the inner member 20, the intermediate tube 36, and the stent 34. It can be appreciated that the deployment sheath 16 is configured to shift between a first position, for example as shown in FIG. 4, where the deployment sheath 16 overlies the stent 34 and a second position where the deployment sheath 16 is proximally retracted to a position substantially proximal of the stent 34. In general, the first position may be utilized during navigation of the system 10 to the appropriate location within a body lumen and the second position may be used to deploy the stent 34.

The deployment sheath 16 may include a flared portion 40 where the outer diameter of the deployment sheath 16 is increased. In the flared portion 40, the thickness of the tubular wall of the deployment sheath 16 may or may not be increased. The flared portion 40 may be desirable for a number of reasons. For example, the flared portion 40 may allow the deployment sheath 16 to have an adequate inner dimension that is suitable so that deployment sheath 16 may be disposed about the stent 34 and the bumper 38.

In at least some instances, the deployment sheath 16 may include a reinforcing member 42 embedded or otherwise included therewith. The reinforcing member 42 may have any number of a variety of different configurations. For example, the reinforcing member 42 may include a braid, coil, mesh, combinations thereof, or the like, or any other suitable configuration. In some instances, the reinforcing member 42 may extend along the entire length of the deployment sheath 16. In other instances, the reinforcing member 42 may extend along one or more portions of the length of the deployment sheath 16. For example, the reinforcing member 42 may extend along the flared portion 40.

The deployment sheath 16 may also include a radiopaque marker or band 44. In general, the marker band 44 may be disposed adjacent to the distal end 46 of the deployment sheath 16. One or more additional marker bands 44 may be disposed along other portions of the deployment sheath 16 or other portions of the system 10. The marker band 44 may allow the distal end 46 of the deployment sheath 16 to be fluoroscopically visualized during advancement of the system 10 and/or deployment of the stent 34.

FIG. 4 also illustrates the distal end 46 of the deployment sheath 16 abutting the proximal ridge 30. In this configuration, the stent 34 can be flushed (e.g., to remove air bubbles) by infusing fluid through the inner member 20 and through the ports 24. Because of the flats 32, fluid may be allowed to be flushed out of the deployment sheath 16 by passing through the gaps formed between the inner member 20 and the deployment sheath 16 at the flats 32.

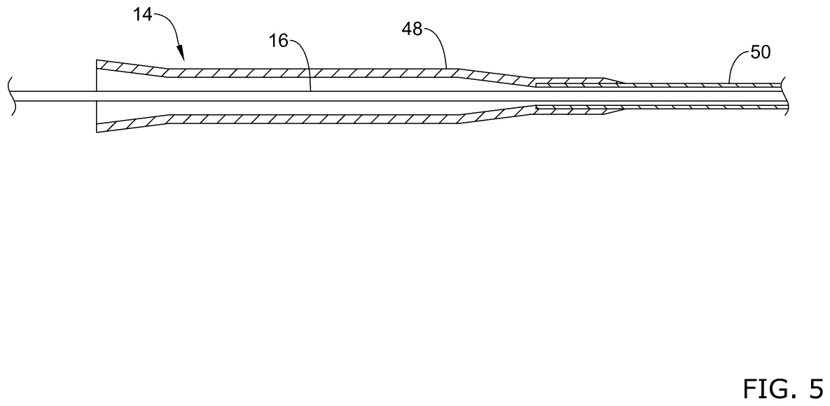

FIG. 5 illustrates a distal portion 48 of the handle 14. Here it can be seen that the handle 14 is attached to an outer member 50. The outer member 50 may be disposed about the deployment sheath 16 and extend along a portion of the length of the deployment sheath 16. Thus, along at least a portion of the length of the system 10, the system 10 may include four tubular structures that may be coaxially arranged-namely the outer member 50, the deployment sheath 16, the intermediate tube 36, and the inner member 20. In at least some embodiments, the outer member 50 may provide the system 10 with a number of desirable benefits. For example, the outer member 50 may include or otherwise be formed from a lubricious material that can reduce friction that may be associated with proximally retracting the deployment sheath 16. In addition, the outer member 50 may comprise a surface that can be clamped or otherwise locked so that the position of the system 10 can be maintained without negatively impacting the retraction of the deployment sheath 16 (which might otherwise be impacted if the deployment sheath 16 was to be clamped). Numerous other desirable benefits may also be achieved through the use of the outer member 50.

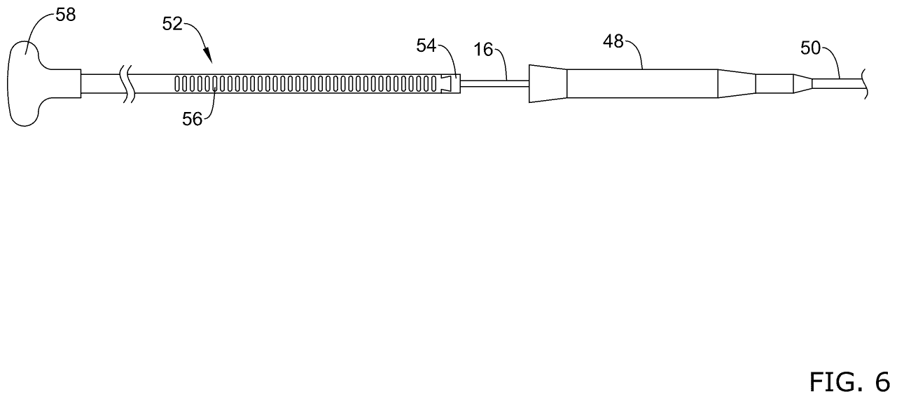

The deployment sheath 16 may pass proximally through the outer member 50 and extend proximally back within the handle 14. The intermediate tube 36 and the inner member 20 both also extend back within the handle 14 and are disposed within the deployment sheath 16. The proximal end of the deployment sheath 16 may be attached to a rack member 52 with a fastener or clip 54 as illustrated in FIG. 6. Thus, it can be appreciated that proximal movement of the rack member 52 may result in analogous proximal movement of the deployment sheath 16. The rack member 52 may include a plurality of teeth or gears 56. In practice, the teeth 56 may be configured to engage with corresponding teeth or gears (not shown) on the thumbwheel 18. Consequently, rotation of the thumbwheel 18, via gearing thereof with the gears 56, can be utilized to proximally retract the rack member 52 and, thus, the deployment sheath 16. Other structural arrangements may be utilized to accomplish proximal retraction of the rack member 52 through the actuation of the thumbwheel 18 or any other suitable actuation member.

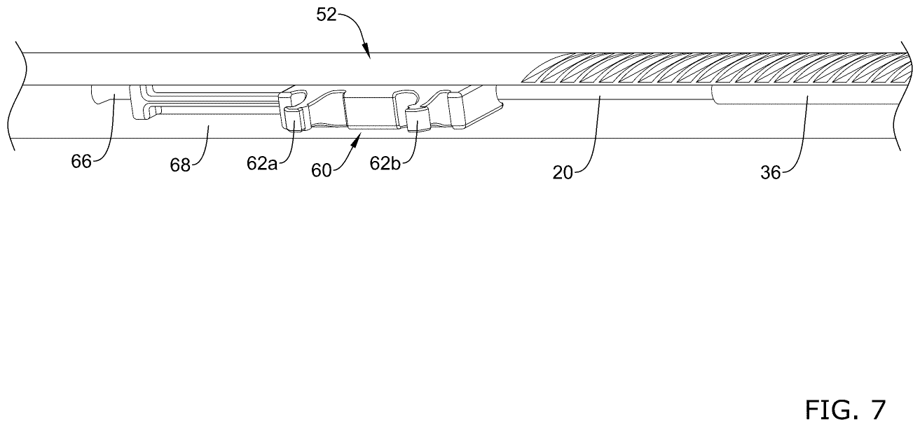

A pull grip 58 may be coupled to the rack member 52. When properly assembled, the main body of the rack member 52 may be disposed within handle 14 and the pull grip 58 may be disposed along the exterior of the handle 14. The rack member 52 may have a slot or groove 68 formed therein (not shown in FIG. 6, can be seen in FIG. 7). The slot 68 may extend the length of the rack member 52, including extending along the pull grip 58. Because the pull grip 58 may be generally located near the proximal end of the inner member 20, the flared shape of the pull grip 58 and the orientation of the slot 68 may allow the pull grip 58 to function as a guidewire introducer or funnel that may assist a clinician in placing, holding, removing, and/or exchanging a guidewire extending through the inner member 20.

In order to properly deploy the stent 34, the various components of the system 10 may need to work in concert so that relative motion of the deployment sheath 16 can be accomplished relative to the inner member 20. In addition, to improve the accuracy of deployment, the intermediate tube 36 may need to be configured so as to provide the desired longitudinal support necessary to limit proximal movement of the stent 34. In at least some embodiments, the proper configuration of these structures may be maintained, at least in part, through the use of a clip member 60 as illustrated in FIG. 7.

In general, the clip member 60 is disposed within the handle 14 and is configured to be secured along the interior of the handle 14. Accordingly, the clip member 60 allows the longitudinal position of one or more portions of the system 10 to be fixed relative to the handle 14. In order to secure the clip member 60 to the handle 14, the clip member 60 may include one or more fasteners or legs 62a/62b. For example, the handle 14 may have one or more slots, grooves, openings, or the like that are configured to seat the legs 62a/62b such that the relative position of the clip member 60 relative to the handle 14 is fixed. In some embodiments, the clip member 60 may be configured to “snap in” to the handle 14. This may desirably simplify manufacturing.

The orientation of the clip member 60 may be such that it is positioned near one or more structures of the system 10. In at least some embodiments, the clip member 60 may be configured so that at least a portion thereof is positioned within the slot 68 of the rack member 52. This may desirably place the clip member 60 near the inner member 20 and the intermediate tube 36 (which may also extend through the slot 68) such that the clip member 60 can be associated therewith. As such, the clip member 60 may aid in maintaining the relative position of one or more structures of the system 10 so that the stent 34 can be accurately deployed. For example, the clip member 60 may include one or more tubular portions that the inner member 20 may pass through and the inner member 20 may optionally include a flared proximal end 66 that may substantially prevent the inner member 20 from moving distally beyond the tubular portion(s). In some embodiments, the clip member 60 may include a flared region or end (not shown), which may facilitate entry of a guidewire into the clip member 60 and/or the inner member 20.

When the stent 34 is deployed, a clinician may actuate the actuation thumbwheel 18. Because of the association of the thumbwheel 18 with the rack member 52, relative rotation of the thumbwheel 18 causes proximal movement of the deployment sheath 16. As the deployment sheath 16 proximally retracts, the stent 34 is “uncovered” and (if the stent 34 is a self-expanding stent) can expand within the body lumen.

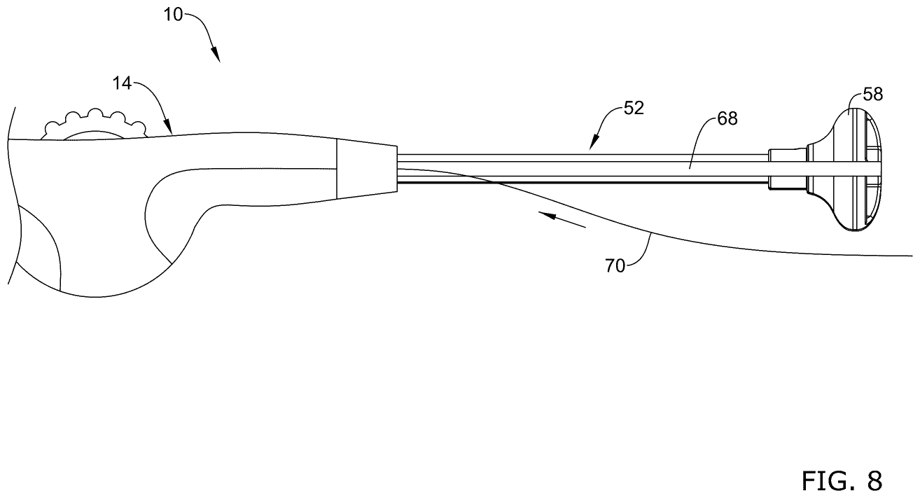

During some interventions, it may be desirable to exchange the system 10 for a different system, catheter, or the like and/or otherwise remove the system 10 from the guidewire 70 (e.g., after deployment of the stent 34). The configuration of the rack member 52, for example including the slot 68, may allow the guidewire 70 to project radially out from the slot 68 in the rack member 52 as depicted in FIG. 8. This allows for a clinician to have access to the guidewire, for example so that device exchanges can be performed.

In some instances, an intervention may generate a number of forces along the shaft 12. For example, forces may be applied to the intermediate tube 36 during proximal retraction of the deployment sheath 16. Such forces may include compressive forces that could cause the intermediate tube 36 to bow, buckle radially outward, and/or otherwise deform. If such deformation occurs, the intermediate tube 36 may be less likely to provide support to the stent 34, which could lead to the delivery problems such as inaccuracy. Disclosed herein are stent delivery systems with improved and/or enhanced delivery accuracy.

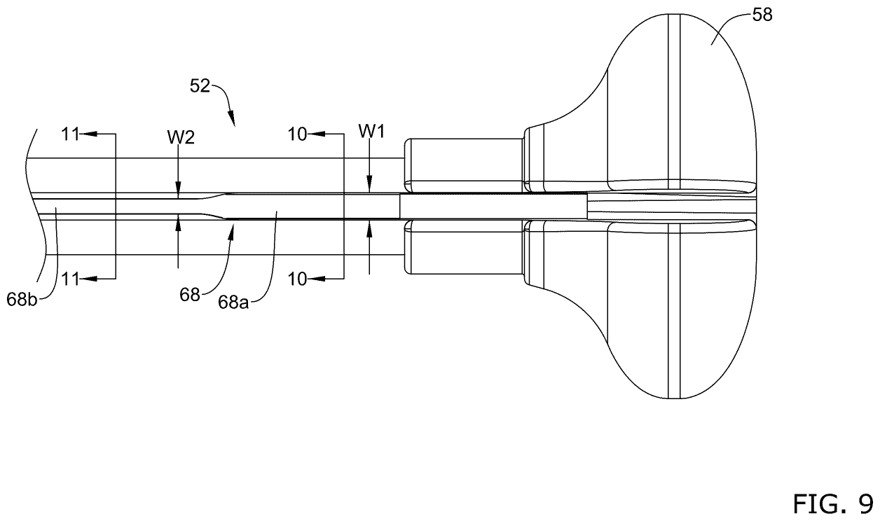

One structural feature that can help reduce deformation of, for example, the intermediate tube 36, is the rack 52. FIG. 9 illustrates the rack 52 and, more particularly, the axial slot 68 formed therein. Here it can be seen that the slot 68 may have a first or proximal portion 68a and a second portion 68b. The first portion 68a may have a first slot width W1. The second portion 68b may have a second slot width W2. In at least some instances, the first slot width W1 and the second slot width W2 are different from one another. In other instances, the first slot width W1 and the second slot width W2 are substantially the same (e.g., the axial slot 68 has a substantially constant slot width).

In some instances, the first portion 68a of the axial slot 68 may be disposed adjacent to the pull handle 58. The second portion 68b may also be considered to be positioned adjacent to the pull handle 58. In some instances, the first portion 68a is disposed between the second portion 68b and the pull handle 58. Other arrangements are contemplated.

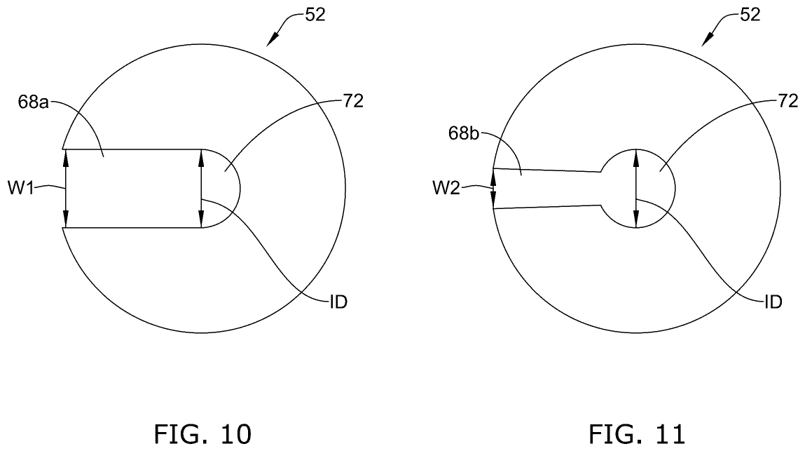

FIGS. 10-11 are cross-sectional views of the rack 52. Here it can be seen that the rack 52 may have a central bore 72 formed therein. The central bore 72 may have or otherwise define a bore or inner diameter ID. In some instances, the first slot width W1 may be substantially equal to the inner diameter ID. It can be appreciated that the intermediate shaft 36 is configured to extend within the central bore 72. Accordingly, the intermediate shaft 36 may have an outer diameter that is less than the inner diameter ID, although the outer diameter may relatively closely approximate the inner diameter in some instances. Thus, the first slot width W1 may be considered to be greater than or equal to the outer diameter of the intermediate shaft 36. By virtue of the first slot width W1 having a relatively large size, it may be relatively easy for a user to grasp devices within the axial slot 68 such as the guidewire 70 (see also, for example, FIG. 8). In addition, the size of first slot width W1 may allow for the clip member 60 to engage structures within the rack 52 (e.g., structures extending within the axial slot 68) such as the inner member 20.

The second slot width W2 may be less than the inner diameter ID. In some of these and in other instances, the second slot width W1 is less than the outer diameter of the intermediate shaft 36. This may be desirable for a number of reasons. For example, by virtue of the second slot width W2 being less than the outer diameter of the intermediate shaft 36, the intermediate shaft 36 may be substantially held or contained within the axial slot 68. In other words, the intermediate shaft 36 may be less likely to bow, buckle radially outward, and/or otherwise deform, for example when subjected to compressive forces. Because of this, the intermediate shaft 36 may be more likely to maintain an unbent configuration and, thus, provide desirable support to the stent 34. This may help to improve delivery accuracy and/or reduce the likelihood that the stent 34 shifts in position during deployment.

In at least some instances, the axial slot 68 may be understood to extend from the central bore 72 to the outer surface or exterior of the rack 52. In some instances, the width of the axial slot 68 may remain constant between the central bore 72 and the exterior of the rack 52. This may be true for the first portion 68a of the axial slot 68, the second portion 68b of the axial slot 68, or both. In other instances, the width of the axial slot 68 may change/vary between the central bore 72 and the exterior of the rack 52. For example, the width of the axial slot 68 may increase as the axial slot shifts radially outward. This may be true for the first portion 68a of the axial slot 68, the second portion 68b of the axial slot 68, or both.

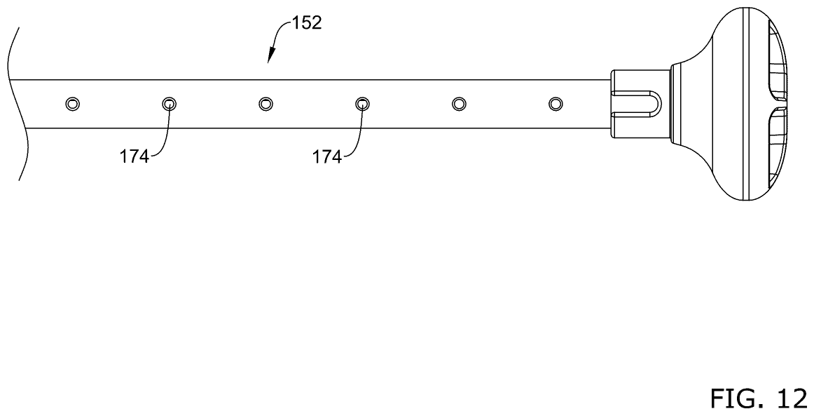

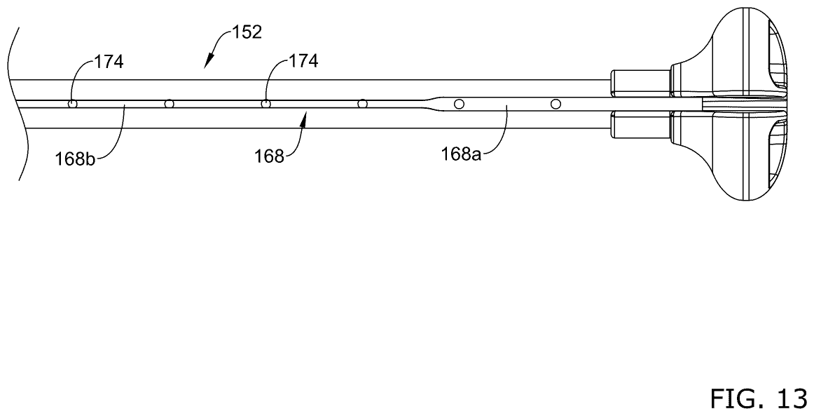

FIGS. 12-13 illustrate another example rack 152 that may be similar in form and function to other rack disclosed herein. In this example, the rack 152 may include molding apertures 174 formed along the length thereof. The molding apertures 174 may make it easier to mold the rack 152 and/or otherwise simply manufacturing. For example, the molding apertures 174 may help to reduce material strain during manufacturing (e.g., molding) of the rack 152 and/or may make it such that a post annealing step/process is not necessary. In some of these and in other instances, the molding apertures 174 may also form pre-determined or intentional break points along the rack 152 that allow for the rack 152 to be broken off after deployment of the stent 34. the molding apertures 174 taking the form of pre-determined or intentional break points along the rack 152 may be similar that what is described in U.S. Pat. No. 11,013,627, the entire disclosure of which is herein incorporated by reference. The rack 152 may also include an axial slot 168 similar in form and function to the axial slot 68. For example, the axial slot 168 may include a first section 168a and a second section 168b.

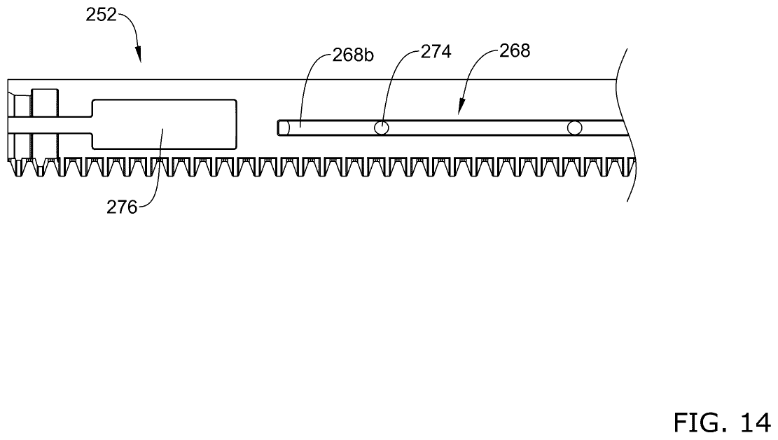

FIG. 14 illustrates another example rack 252 that may be similar in form and function to other rack disclosed herein. In this example, the rack 252 may include an axial slot 268. The axial slot 268 may include a first portion (not shown) and a second portion 268b. One or more molding apertures 274 may be formed in the rack 252. The rack 252 may also include a sidewall opening 276. The sidewall opening 276 may be configured to allow the deployment sheath 16 to be mechanically secured to the rack 252. For example, the deployment sheath 16 may be threaded through the distal end of the rack 252 and then through the sidewall opening 276. If desired, a structure such as a cuff or sleeve maybe bonded to the deployment sheath 16. The deployment sheath 16 may then be advanced until the cuff or sleeve is seated within the sidewall opening 276 in a manner that axially secures the deployment sheath 16 relative to the rack 252 while permitting the deployment sheath 16 to rotate relative to the rack 252. Some example structures for mechanically securing the deployment sheath 16 with the rack 252 may include those disclosed in U.S. Pat. No. 11,602,447, the entire disclosure of which is herein incorporated by reference.

The materials that can be used for the various components of the system 10 may include those commonly associated with medical devices. For simplicity purposes, the following discussion makes reference to the shaft 12 and other components of the system 10. However, this is not intended to limit the devices and methods described herein, as the discussion may be applied to other similar tubular members and/or components of tubular members or devices disclosed herein.

The shaft 12 and/or other components of the system 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the system 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system 10 to achieve the same result.

In some embodiments, a degree of Magnetic Resonance Imaging (MRI) compatibility is imparted into the system 10. For example, the system 10, or portions thereof, may be made of a material that does not substantially distort the image and create substantial artifacts (e.g., gaps in the image). Certain ferromagnetic materials, for example, may not be suitable because they may create artifacts in an MRI image. The system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A stent delivery system, comprising:
an elongate shaft including an inner member having a stent receiving region, a deployment sheath slidably disposed along the inner member, and an intermediate shaft disposed between the inner member and the deployment sheath;
wherein the intermediate shaft has an outer diameter;
a handle coupled to the elongate shaft;
a rack member coupled to the deployment sheath, wherein at least a region of the rack member is configured to extend within the handle;
wherein the rack member has an axial slot formed therein; and
wherein at least a portion of the axial slot has a width that is less than the outer diameter of the intermediate shaft.

2. The stent delivery system of claim 1, wherein the rack member includes a plurality of teeth.

3. The stent delivery system of claim 1, further comprising a pull handle coupled to the rack member.

4. The stent delivery system of claim 3, wherein the pull handle is disposed proximal of a proximal end of the handle.

5. The stent delivery system of claim 3, wherein the portion of the axial slot having the width that is less than to the outer diameter of the intermediate shaft is disposed adjacent to the pull handle.

6. The stent delivery system of claim 3, wherein a proximal portion of the axial slot having a second width that is greater than or equal to the outer diameter of the intermediate shaft is disposed between the pull handle and the portion of the axial slot having the width that is less than the outer diameter of the intermediate shaft.

7. The stent delivery system of claim 1, wherein the rack member has a central bore having a bore diameter and wherein the width of the portion of the axial slot is less than to the central bore.

8. The stent delivery system of claim 1, wherein the rack member has one or more molding apertures formed therein.

9. The stent delivery system of claim 1, wherein the rack member has a sidewall opening formed therein that is configured to allow the deployment sheath to be mechanically secured to the rack member.

10. The stent delivery system of claim 1, wherein the elongate shaft includes an outer shaft disposed along a section of an outer surface of the deployment sheath.

11. A stent delivery system, comprising:
an inner member having a stent receiving region;
a deployment sheath slidably disposed along the inner member;
a rack member coupled to the deployment sheath, the rack member having an axial slot formed therein;
wherein the axial slot has a first portion having a first slot width and a second portion having a second slot width different from the first slot width; and
a handle disposed over at least a portion of the rack member.

12. The stent delivery system of claim 11, further comprising an intermediate shaft disposed between the inner member and the deployment sheath.

13. The stent delivery system of claim 12, wherein the first slot width is greater than or equal to an outer diameter of the intermediate shaft.

14. The stent delivery system of claim 12, wherein the second slot width is less than an outer diameter of the intermediate shaft.

15. The stent delivery system of claim 11, further comprising a pull handle coupled to the rack member and disposed proximal of a proximal end of the handle.

16. The stent delivery system of claim 15, wherein the first portion of the axial slot is disposed adjacent to the pull handle.

17. The stent delivery system of claim 15, wherein the first portion of the axial slot is disposed between the pull handle and the first portion of the axial slot.

18. The stent delivery system of claim 11, wherein the rack member has a central bore having a bore diameter and wherein the second slot width of the axial slot is less than the central bore.

19. A stent delivery system, comprising:
an inner member having a stent receiving region;
a deployment sheath slidably disposed along the inner member;
an intermediate shaft disposed between the inner member and the deployment sheath;
a rack member coupled to the deployment sheath, the rack member having an axial slot formed therein; and
wherein the axial slot has a first portion having a first slot width and a second portion having a second slot width different from the first slot width.

20. The stent delivery system of claim 19, wherein the second slot width is less than an outer diameter of the intermediate shaft.
